(19) United States
(12) Patent Application Publication
(10) Pub. No.: US 2015/0191396 A1
(43) Pub. Date: Jul. 9, 2015

(54) CERAMIC COMPONENT FOR FUSING VERTEBRAL BODIES

(71) Applicant: CeramTec GmbH, Plochingen (DE)

(72) Inventors: Alfons Kelnberger, Rothenbach (DE); Fiona Neumann, Lauf (DE); Heinrich Wecker, Eckental (DE); Frank Ziermann, Berlin (DE)

(21) Appl. No.: 14/413,921

(22) PCT Filed: Jul. 9, 2013
(86) PCT No.: PCT/EP2013/064440

§ 371 Date: Jan. 9, 2015

(30) Foreign Application Priority Data
Jul. 11, 2012 (DE) 10 2012 212 138.2

(51) Int. Cl. C04B 35/10; C04B 35/64; A61L 24/02

(52) U.S. Cl. CPC C04B 35/10; A61L 24/02; C04B 35/64

(57) ABSTRACT

Oxide ceramic components for fusing vertebral bodies and methods for producing the components.

The invention relates to ceramic components, In particular implants, for fusing vertebral bodies, and to methods for producing these ceramic components.

Components for fusing vertebral bodies based on metal materials such as e.g. tantalum or titanium are known. A drawback of these metal materials is for instance a high risk of infection. Moreover, metal implants may be medically contraindicated if there is an allergy or hypersensitivity to metals or metal-like materials. Metal abrasion may have negative effects on the human organism. Artifacts from metal implants may greatly impede imaging in medical diagnostics.

Components based on plastics, such as e.g. highly cross-linked PE materials or PEEK, are also known. Disadvantages of the plastics are that, e.g., the mechanical properties may be unsatisfactory, which may lead, e.g., to tips or other parts of the component breaking off, for instance during insertion. Moreover, components or implants based on plastics cannot be imaged, or cannot be adequately imaged, with many current imaging methods, e.g. MRI and X-rays, so that for instance specific markers must be used.

Ceramic components based on silicon nitride are also known. However, this class of materials was developed with excellent high temperature properties in mind—for instance for mechanical machining of metal components for the automobile industry—and in terms of the properties required for this application, such as strength, hardness, and long-term stability, is more in the mid-range compared to other high-performance ceramic materials based on oxide systems. In addition, this material is a relatively complicated material containing needle-shaped silicon nitride, which is embedded in a glass matrix. Therefore, sintering the material is complex. Moreover, mechanical machining processes such as grinding and polishing are very demanding and difficult because the material is very hard and nonhomogeneous. In addition, components produced from silicon nitride have a rather dark coloring—gray to black—which in the medical field is less accepted for reasons of appearance alone. All of these disadvantages lead to increased costs in producing the components, which represents a further drawback.

The object of the invention is therefore to provide a method for producing a component for fusing vertebral bodies, and to provide a component for fusing vertebral bodies that avoids the aforesaid disadvantages and in particular has adequate strength, hardness, and long-term stability. The object of the invention is further to provide a method that imposes as few special requirements as possible on the machining process.

The object is achieved using a component for fusing vertebral bodies in accordance with claim 1 and a method for producing this component in accordance with claim 8.

The component according to the invention for fusing vertebral bodies consequently is made of an oxide ceramic. The produced products have the advantages of oxide ceramics. Oxide ceramics are distinguished in particular by high durability in, and good tolerance with, body media. Oxide ceramics have good biocompatibility and do not cause any allergic reactions.

Consequently, the invention includes a ceramic component for fusing vertebral bodies, especially in the area of the human vertebral column. In accordance with one preferred embodiment of the invention, the component is based on oxide ceramic material systems, including:
Zirconium oxide-toughened aluminum oxide (ZTA) and all refined ZTA systems based thereon;
Zirconium oxide ceramic, especially yttrium-stabilized zirconium oxide (3Y-TZP);
Cerium-stabilized zirconium oxide (Ce-TZP), in which the tetragonal phase of the zirconium oxide is stabilized by cerium oxide;
All other composite materials based on zirconium oxide, wherein the dispersoid composite components may be based on aluminates, and also other stabilizers from the group of rare earths may be used, such as Gd and Sm, e.g.

A few of the materials in question shall be briefly described in the following.
ZTA: Aluminum oxide is the base material. Compared to conventional ZTAs, its strength and fracture toughness are doubled by adding oxides and ceramic platelets. This makes it possible to implement components in additional sizes and for new applications.

ZTA may be produced, for instance, from materials having the following composition: 72 to 82 wt. % Al₂O₃, 28 to 18 wt. % ZrO₂, 0 to 1 wt. % Cr₂O₃, 0 to 6 wt. % Y₂O₃ relative to the ZrO₂ content, 0 to 2 wt. % SrO, 0 to 0.5 wt. %, TiO₂, and 0 to 0.5 wt. % MgO.

One preferred embodiment of the invention includes a material having the following composition: 72.65 to 74.54 wt. % Al₂O₃, 24.0 to 25.5 wt. % ZrO₂, 0.5 to 0.65 wt. % Y₂O₃ relative to the Al₂O₃ content, 0.26 to 0.35 Cr₂O₃, and 0.70 to 0.85 wt. % SrO.

In accordance with another preferred embodiment of the invention, the component has the following composition: 70 to 90 wt. % Al₂O₃:Cr (aluminum oxide with chromium doping), 12 to 22 wt. % ZrO₂:Y (yttrium-stabilized zirconium oxide) and 1 to 5 wt. % SrAl₁₂₋ₓCrₓO₁₉ (strontium aluminate with variable chromium doping, where x is preferably between 0.0007 and 0.045).
3Y zirconium oxide, 3Y-TZP: among ceramic materials, zirconium oxide (ZrO₂) has the highest fracture toughness. So-called stabilizers such as yttrium oxide are needed for producing solid bodies; a strength of more than 1600 MPa may be obtained with this stabilizer. Similarly as for ZTA, crack propagation is blocked by the so-called transformation toughening. It is also possible to produce sharp edges from zirconium oxide, which makes it an ideal material for producing components with self-cutting threads, which is already used in dentistry, far instance.
Ce zirconium oxide; Ce-TZP: cerium oxide (Ce₂O₃) may likewise be used as a stabilizer for zirconium oxide. With an optimum structure, this material attains even higher fracture toughness than 3Y-TZP. It even permits limited plastic deformation, similar to metals. On the other hand, its strength and hardness are lower than for 3Y zirconium oxide. With its elasticity, which is likewise dearly higher, this material is a particular preferred choice for producing a component according to the invention.

Naturally, all refinements and all variants of these material classes are also in principle suitable for the component according to the invention, such as e.g. composite materials based on yttrium-stabilized zirconium oxide with strontium hexaaluminate as the secondary, dispersoid, toughening phase in the structure (SHYTZ).

The geometry of the component is matched to the anatomy of the human vertebral body. The component is seated between two vertebral bodies and replaces all or part of the intervertebral disk. In a first phase of its residence in the human body, the component holds the vertebral bodies at a distance and in an anatomically correct position solely by virtue of its mechanical properties. In a second phase, the component promotes fusing, and thus the growing together of the two vertebral bodies between which it is inserted.

For the first phase, the so-called primary stability immediately following the surgery and prior to osseointegration is important. This may be attained, for instance, in that morphological moldings that ensure slip-proof connection of the adjacent vertebrae are provided on the top and bottom sides of the component, which are in contact with the adjacent vertebrae. Such morphological moldings may be pointed, pyramidal, or knob-shaped structures, for instance. By means of these structures, the components can hook onto the vertebral bodies, or the component is fixed in the position in which it was inserted.

Mechanical stability is assured by the excellent mechanical material properties of the aforesaid material classes. Ideally, the component is embodied in an annular or banana shape, wherein the geometry and size are adapted to the different areas of the vertebral column (e.g. cervical or lumbar area). In addition, the shape of the component plays an important role in the insertion or implantation in the human body. Different component shapes that are known per se to the person skilled in the art are required for different implantation methods.

In accordance with one advantageous refinement of the invention, the component has an outer, solid or completely ceramic part that is extremely well suited for the mechanical, biological, and chemical requirements during implantation and also while it remains in the human body.

The component preferably also has an inner part that is configured in such a way that conditions are optimal for human bone cells (e.g. osteoblasts) or cells that are necessary for the formation of human bone tissue (ossification). The goal here is complete bony integration into the human vertebral column, so-called osseointegration.

In accordance with a first variant, this inner part may be hollow; that is, it may be merely an empty area free of ceramic, that may be used for introducing the body's own, or autologous, bone material, preferably together with known substances that promote ossification.

In a second variant, this inner part may also be porous; that is, it may be embodied as a porous, preferably ceramic structure. This porous structure may particularly preferably be implemented on the basis of the same ceramic material as the outer part. It has been found that the following properties of an implant have a positive effect on ossification:
Porosities between 50 and 99%, preferably between 70 and 85%;
Interconnectivity; i.e., at least some of the individual pores are connected to one another;
Pore sizes between 100-1000 μm, preferably between 500 to 800 μm.

The structure of the inner part may be produced by means of different methods, in particular directly during the process of producing the ceramic component, or separately by subsequently introducing the inner part into the outer part.

The direct production processes include, e.g., a two-component injection molding process in which preferably the outer part is first cast in a mold and then the inner part is cast, especially by suitably modifying the mold. The two parts are subsequently co-sintered and undergo final machining.

The inner part may also be produced as a foam-like structure, for instance by freeze direct foaming. In accordance with another alternative, organic materials may be added to a ceramic slurry and subsequently burned out so that pores remain.

During separate production and subsequent introduction of the inner part into the outer part, the two structures, the outer part and inner part, are molded and sintered independently of one another and are not combined until a second step, preferably by mechanical means. Direct molding processes may for instance be used during separate production of the inner part. In this case, polyurethane foams are suitable which, after appropriate pretreatment to produce a suitable structure, are impregnated with ceramic slurry and then burned out. For the purpose of direct molding, biomimetic methods may also be used which per se have a trabecular bone structure or a similar structure. For instance, organic materials like bamboo are suitable for this purpose.

It is also possible to produce the structures of the inner part in a defined fashion using generative methods, for instance by means of printing methods or dispense plotting. Printing methods have the advantage that the geometry of the individual pores of the porous structure may be defined and produced periodically. Thus, it is possible from a technical standpoint to design, develop, and produce an optimal scaffold for the biological-chemical processes during ossification.

For additionally increasing osseoinductivity or bioactivity, the structures of the inner and/or outer part may be coated with common functional coatings such as e.g. hydroxyapatite or tricalcium phosphate or other calcium phosphates that promote osseointegration, e.g. Fillings based on bioglass ceramic materials that have a high proportion of SiO₂, CaO, P₂O₅, and/or K₂O are also suitable for this purpose. The component or just only the Inner or outer part may also be coated with this material.

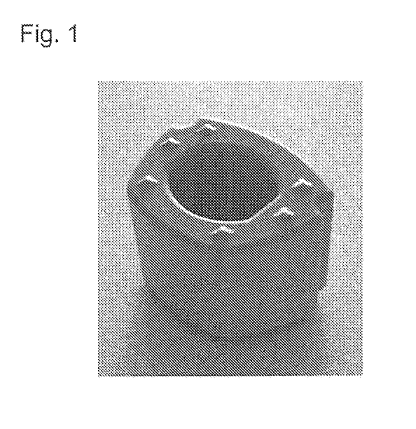

FIG. 1 depicts one possible embodiment of a ceramic component according to the invention for fusing vertebral bodies. The implant has a stable solid outer area with pyramidal structures on the top and bottom for fixing against displacement on the adjacent vertebral bodies. The inner part is designed as a hollow space that may be filled for instance with autologous bone to promote osseointegration. The component is made of a zirconium oxide-toughened aluminum oxide ceramic.

1.-15. (canceled)

16. A ceramic component for fusing vertebral bodies, wherein the component comprises an oxide ceramic.

17. A ceramic component in accordance with claim 16, wherein the oxide ceramic comprises an ceramic selected from the group consisting of an aluminum oxide ceramic and a zirconium oxide ceramic as the secondary, dispersoid, toughening phase in the structure.

18. A ceramic component in accordance with claim 16, wherein the component has an outer and an inner part, wherein the outer part is preferably solid and/or the inner part is preferably hollow or porous.

19. A ceramic component in accordance with claim 16, wherein the porosity of the inner part is between 50 and 99%.

20. A ceramic component in accordance with claim 16, wherein a top side and a bottom side of the component that are in contact with the cover plates of the adjacent vertebrae have morphological moldings.

21. A ceramic component in accordance with claim 18, wherein the hollow inner part of the component has a filling based on a bioglass ceramic material comprising at least one member selected from the group consisting of SiO₂, CaO, P₂O₅ and K₂O.

22. A ceramic component in accordance with claim 16, wherein the ceramic component is provided with a functional coating, especially hydroxyapatite, tricalcium phosphate, or other calcium phosphates.

23. A method for producing a ceramic component for fusing vertebral bodies, wherein the ceramic component is produced from an oxide ceramic.

24. A method in accordance with claim 23, wherein an aluminum oxide ceramic, especially a zirconium oxide-toughened aluminum oxide, a zirconium oxide ceramic, especially an yttrium-stabilized zirconium oxide or cerium-stabilized zirconium oxide or an yttrium-stabilized zirconium oxide with strontium hexaaluminate is produced as the secondary, dispersoid, toughening phase in the structure.

25. A method in accordance with claim 23, wherein the ceramic component is produced with at least one of a solid outer part and a hollow or porous inner part.

26. A method in accordance with claim 25, wherein the ceramic component is produced by means of a two-component injection molding process, wherein preferably first the outer part is cast in a mold, then the inner part is cast by suitably modifying the mold, and the two parts are subsequently co-sintered.

27. A method in accordance with claim 25, wherein direct molding methods are used for producing the porous inner part.

28. A method in accordance with claim 25, wherein organic material is added to a ceramic slurry or is impregnated with a slurry and the organic material is subsequently burned out.

29. A method in accordance with claim 25, wherein biomimetic methods are used for molding the porous structure of the inner part, wherein the porous structure comprises a material that has a trabecular structure.

30. A method in accordance with claim 25, wherein the porous inner part is produced via freeze direct foaming or via a generative method.

31. A ceramic component in accordance with claim 16, wherein the oxide ceramic comprises a member selected from the group consisting of zirconium oxide-toughened aluminum oxide, an yttrium-stabilized zirconium oxide, a cerium-stabilized zirconium oxide, and an yttrium-stabilized zirconium oxide with strontiumhexaaluminate as the secondary, dispersoid, toughening phase in the structure.

32. A method according to claim 29, wherein the material that has a trabecular structure is bamboo.

33. A method in accordance with claim 25, wherein the porous inner part is produced via a printing method or by of dispense plotting.

34. A ceramic component in accordance with claim 16, wherein the ceramic component is provided with a functional coating selected from the group consisting of hydroxyapatite, tricalcium phosphate and another calcium phosphates.

35. A ceramic component in accordance with claim 18, wherein the porous inner part is coated with a bioglass ceramic material comprising at least one member selected from the group consisting of SiO₂, CaO, P₂O₅ and K₂O.

36. A ceramic component in accordance with claim 20, wherein the morphological moldings are pointed, pyramidal or knob-shaped.

37. A ceramic component in accordance with claim 18, wherein the porosity of the inner part is between 70 and 85%.

38. A ceramic component in accordance with claim 18, wherein the inner part is porous and wherein pores of the inner porous part are interconnective.

39. A ceramic component in accordance with claim 18, wherein the inner part is porous and wherein the pores have a diameter between 100 and 1000 μm.

40. A ceramic component in accordance with claim 18, wherein the inner part is porous and wherein the pores have a diameter between 500 to 800 μm.

41. A ceramic component in accordance with claim 18, wherein the outer part is solid.

42. A ceramic component in accordance with claim 18, wherein the inner part is hollow.

43. A ceramic component in accordance with claim 18, wherein the inner part is porous.
